(19) United States
(12) Patent Application Publication
(10) Pub. No.: US 2026/0204371 A1
(43) Pub. Date: Jul. 16, 2026

(54) VACCINATION CERTIFICATE VERIFICATION SYSTEM, VACCINATION CERTIFICATE VERIFICATION METHOD, AND PROGRAM

(71) Applicant: NEC Corporation, Tokyo (JP)

(72) Inventor: Shinichi TORIYAMA, Tokyo (JP)

(73) Assignee: NEC Corporation, Tokyo (JP)

(21) Appl. No.: 19/134,364

(22) PCT Filed: Dec. 9, 2022
(86) PCT No.: PCT/JP2022/045450

§ 371 Date: May 30, 2025

(51) Int. Cl. G16H 10/60

(52) U.S. Cl. CPC G16H 10/60

(57) ABSTRACT

There is provided a vaccination certificate verification system that contribute to making it possible to verify the authenticity of the vaccination certificate. A vaccination certificate verification system includes a vaccination history registration part which registers information identifying a vaccination history in a blockchain and issues a vaccination certificate including information regarding the vaccination history after registration of the vaccination history; and a vaccination certificate verification part which determines that the vaccination certificate is authentic in a case where information identifying the vaccination history included in information regarding the history in the vaccination certificate exists in the blockchain.

### TECHNICAL FIELD

The present invention relates to a vaccination certificate verification system, a vaccination certificate verification method, and a program.

### BACKGROUND ART

There are literatures regarding vaccination against infectious diseases and certificates of immunity, or the like below.

Patent Literature (PTL) 1 relates to a new coronavirus vaccination management system in which iris information of a vaccinated person and identification information of a vaccinated vaccine are linked at the time of vaccination and they are managed as a distribution database utilizing a blockchain technology.

PTL 2 relates to an information processing method which records a user-downloadable document as a vaccination history and a certificate regarding an infectious disease issued by an examination organization in a distributed database including a blockchain.

PTL 3 relates to an information system which includes a storage means for storing an immunity certificate and in which updated information of the immunity certificate is stored as a hash value regarding the immunity certificate when the immunity certificate stored in the storage means is updated.

Non-Patent Literature (NPL) 1 relates to Bitcoin.

### CITATION LIST

### Patent Literature

[PTL 1] Japanese Patent Kokai Publication No. 2022-050286A

[PTL 2] Japanese Patent Kokai Publication No. 2022-037803A

[PTL 3] WO 2021/250857A

### Non-Patent Literature

[NPL 1] S. Nakamoto, “Bitcoin: a Peer-TO-Peer Electronic Cash System”, [online], [searched on Jun. 28, 2022], Internet <URL: https://bitcoin.org/bitcoin.pdf

### SUMMARY

### Technical Problem

The following analysis is given by the inventor.

Under the situation of an infectious disease epidemic, it may be required to present a vaccination certificate to a service provider for receiving service provision. However, if no protection is provided against falsification of a vaccination certificate, it is conceivable that falsification of vaccination certificates will become widespread. In order to adequately prevent falsification of vaccination certificates, it is preferable to be able to verify the authenticity of the vaccination certificate.

It is an object of the present invention to provide a vaccination certificate verification system, a vaccination certificate verification method, and a program that contribute to making it possible to verify the authenticity of the vaccination certificate.

### Solution to Problem

According to a first aspect of the present invention, there is provided a vaccination certificate verification system, comprising:
a vaccination history registration part which registers information identifying a vaccination history in a blockchain and issues a vaccination certificate including information regarding the vaccination history after registration of the vaccination history; and
a vaccination certificate verification part which determines that the vaccination certificate is authentic in a case where information identifying the vaccination history included in information regarding the history in the vaccination certificate exists in the blockchain.

According to a second aspect of the present invention, there is provided a vaccination certificate verification method performed in a vaccination certificate verification system including a vaccination history registration part and a vaccination certificate verification part, comprising:
a step of registering information identifying a vaccination history in a blockchain and issuing a vaccination certificate including information regarding the vaccination history after registration of the vaccination history by the vaccination history registration part; and
a step of determining that the vaccination certificate is authentic in a case where information identifying the vaccination history included in information regarding the history in the vaccination certificate exists in the blockchain by the vaccination history registration part. This method is associated with a certain machine, which is a computer that performs a vaccination certificate verification method.

According to a third aspect of the present invention, there is provided a program, causing a computer to execute a processing of:
registering information identifying a vaccination history in a blockchain; and
determining that the vaccination certificate is authentic in a case where information identifying a vaccination history included in information regarding the history in a vaccination certificate including information regarding the vaccination history issued after registration of the vaccination history exists in the blockchain.

It is to be noted that this program may be recorded on a computer-readable storage medium. The storage medium can be non-transitory one, such as a semiconductor memory, a hard disk, a magnetic recording media, an optical recording media and so on. The present invention can be implemented as a computer program product.

### Advantageous Effects of Invention

According to the present invention, it is possible to provide a vaccination certificate verification system, a vaccination certificate verification method, and a program that contribute to making it possible to verify the authenticity of the vaccination certificate.

### BRIEF DESCRIPTION OF DRAWINGS

FIG. 1 is a diagram illustrating an example of a configuration of a vaccination certificate verification system according to an example embodiment of the present invention.

FIG. 2 is a diagram illustrating an example of a configuration of a vaccination certificate verification system according to a first example embodiment of the present invention.

FIG. 3 is a diagram illustrating an example of an operation of a block generation part of a blockchain according to the first example embodiment of the present invention.

FIG. 4 is a flowchart illustrating an example of a schematic operation of a vaccination certificate verification system according to the first example embodiment of the present invention.

FIG. 5 is a diagram illustrating an example of a configuration of a vaccination certificate verification system according to a second example embodiment of the present invention.

FIG. 6 is a flowchart illustrating an example of a schematic operation of a vaccination certificate verification system according to the second example embodiment of the present invention.

FIG. 7 is a diagram illustrating an example of a configuration of a vaccination certificate verification part according to a third example embodiment of the present invention.

FIG. 8 is a diagram illustrating a configuration of a computer that makes up a vaccination certificate verification part of the present invention.

### DETAILED DESCRIPTION

First, an outline of one example embodiment of the present invention will be described with reference to the drawings. Note, in the following outline, reference signs of the drawings are denoted to each element as an example for the sake of convenience to facilitate understanding and are not intended to limit the present invention to the illustrated modes. Furthermore, the connection lines between blocks in the drawings and the like referred to in the following description include both one-way and two-way directions. A one-way arrow schematically illustrates a principal signal (data) flow and does not exclude bidirectionality.

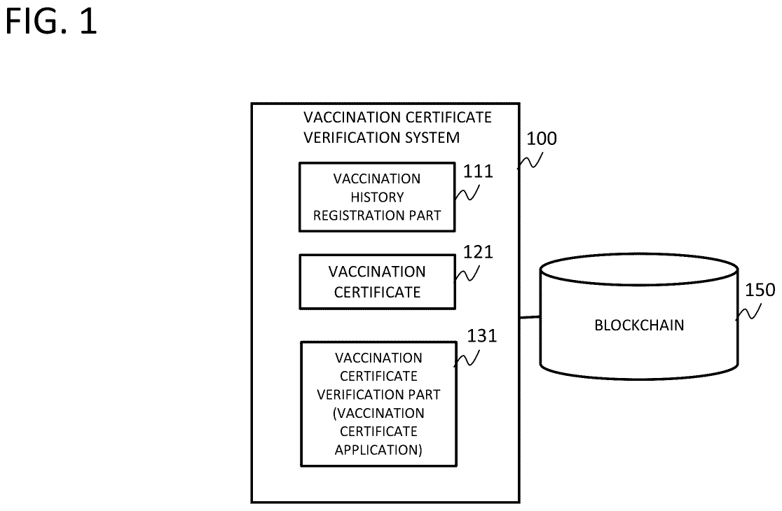

FIG. 1 is a diagram illustrating an example of a configuration of a vaccination certificate verification system according to an example embodiment of the present invention. With reference to FIG. 1, a vaccination certificate verification system 100 is connected to a blockchain (or a distributed ledger) 150 and includes a vaccination history registration part 111, a vaccination certificate 121 issued by the vaccination history registration part 111, and a vaccination certificate verification part 131. The blockchain 150 may be provided and serviced by different entity from the vaccination history registration part 111, the vaccination certificate 121 issued by the vaccination history registration part 111, and the vaccination certificate verification part 131, and may be connected to through a communication line and configured to be accessed from the vaccination certificate verification system 100. Otherwise, the blockchain 150 may be provided in the vaccination certificate verification system 100.

The vaccination history registration part 111 registers information identifying a vaccination history in the blockchain 150 and issues the vaccination certificate 121 including information regarding the vaccination history after registration of the vaccination history.

Because the blockchain 150 registers information identifying a vaccination history as a chain of blocks which are difficult to be falsified, anyone who can access the blockchain 150 can easily access information identifying a vaccination history difficult to be falsified registered in the blockchain 150. Note, as an example, information identifying a vaccination history may be a hash value of the vaccination history although it is not limited thereto. In a case where information identifying a vaccination history may be a hash value of the vaccination history, it is possible to keep the information be confidential from a general viewer.

The vaccination certificate verification part 131 determines that the vaccination certificate 121 is authentic in a case where information identifying the vaccination history included in information regarding the vaccination history in the vaccination certificate exists in the blockchain 150. Note, an example of information identifying a vaccination history may be some kinds of two-dimensional codes, such as a QR code (Registered Trademark) or the like, or codes utilizing other coding methods, but not limited thereto. Otherwise, as an example, information identifying a vaccination history may be information represented by other representation method, such as a URI (Uniform Resource Identifier) or the like. For example, information regarding a vaccination history may be described as a hash value of the vaccination history as it is.

According to one example embodiment of the present invention, it is possible to provide a vaccination certificate verification system, a vaccination certificate verification method, and a program that contribute to making it possible to verify the authenticity of the vaccination certificate.

### First Example Embodiment

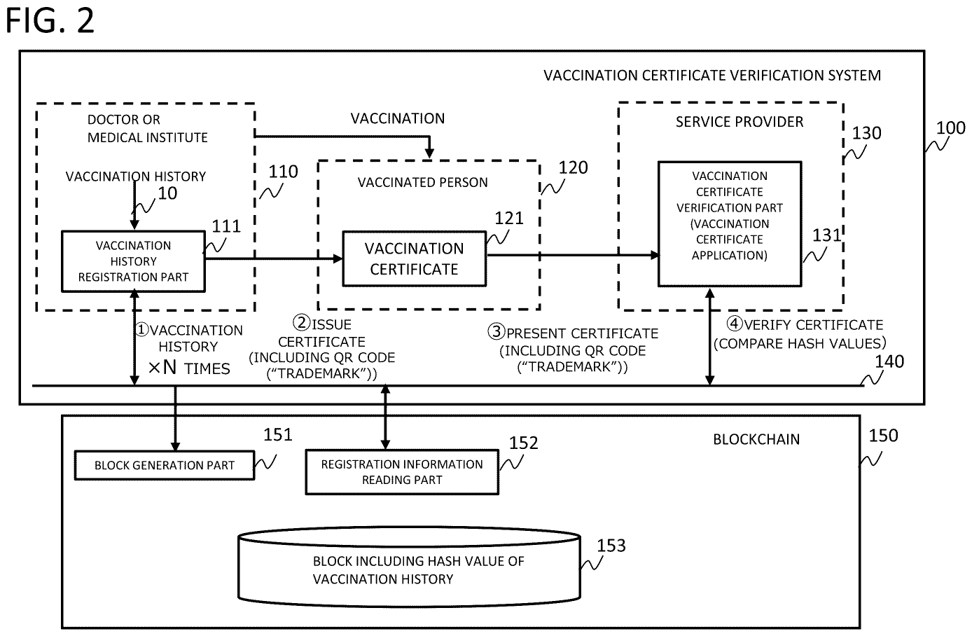

Next, a vaccination certificate verification system according to a first example embodiment of the present invention will be described with reference to drawings. FIG. 2 is a diagram illustrating an example of a schematic configuration of a vaccination certificate verification system according to a first example embodiment of the present invention. In FIG. 2, it is assumed that the components with the same reference signs as those in FIG. 1 are the same, and their description will be omitted. In the following description, although a hash value of the vaccination history is described as information identifying a vaccination history, information identifying a vaccination history different from a hash value of the vaccination history may be used. In the following description, although a code (for example, a QR (Quick Response) code is described as information regarding a vaccination history, for example, information represented by other representation method different from a QR code, such as some kinds of two-dimensional codes, codes utilizing other coding methods, a URI (Uniform Resource Identifier) or the like may be used. Note, the same applies to a second example embodiment and a third example embodiment of the present invention.

With reference to FIG. 2, a vaccination certificate verification system 100 includes a vaccination history registration part 111 at a doctor or medical institution 110, a vaccination certificate 121 issued to a vaccinated person 120 by the vaccination history registration part 111 at the doctor or medical institution 110 which vaccinated to the vaccinated person 120, and a vaccination certificate verification part 131 at a service provider. The vaccination certificate verification system 100 is connected to a blockchain 150 by a network 140. Note, the blockchain 150 may be provided and operated by an entity different from the vaccination history registration part 111, the vaccination certificate 121 issued by the vaccination history registration part 111, and the vaccination certificate verification part 131, and may be connected through a communication line and configured to be accessed by the vaccination certificate verification system 100, or may be provided within the vaccination certificate verification system 100.

The vaccination history registration part 111 calculates a hash value of a vaccination history 10, as information for identifying the vaccination history 10, from the vaccination history 10 of a vaccinated person 120 inputted by a doctor or medical institution 110. Note, instead of a doctor or medical institution 110, an administrative agency or the like may input the vaccination history 10 of the vaccinated person 120 into the vaccination history registration part 111. The vaccination history registration part 111 sends a hash value calculated from a vaccination history 10 of the vaccinated person 120 to the blockchain 150 via the network 140.

Note, the vaccination history registration part 111 can prevent the vaccination history 10 itself of a vaccinated person 120 from being leaked to viewers of a blockchain 150 by sending a hash value calculated from a vaccination history 10 of a vaccinated person 120 to a blockchain 150, even if the vaccination history 10 is recorded in a blockchain.

The blockchain 150 includes a block generation part 151 and a registration information reading part 152. When the block generation part 151 receives a hash value of a vaccination history 10 of a vaccinated person 120 sent from a vaccination history registration part 111, the block generation part 151 generates a block and registers it in a blockchain 150 as a block 153 including a hash value of a vaccination history 10.

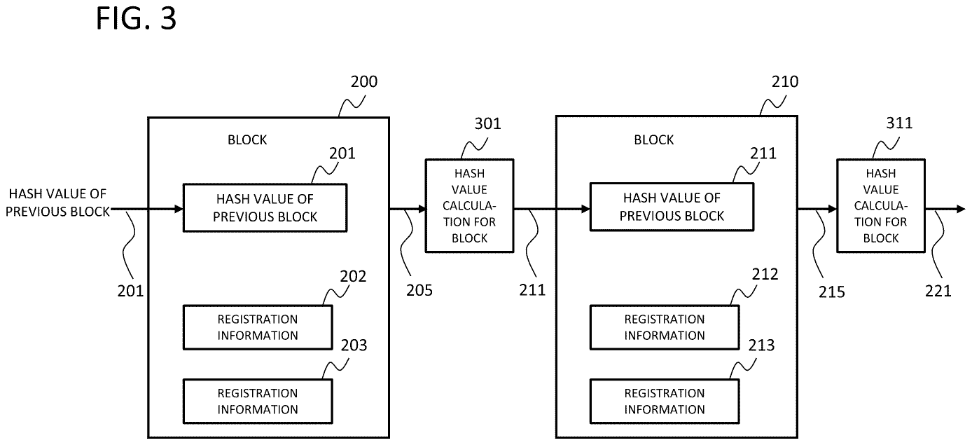

FIG. 3 is a diagram illustrating an example of an operation of a block generation part 151 of a blockchain 150 according to the first example embodiment of the present invention. The block generation part 151 generates a block 200 which includes one or more pieces of received registration information (transactions) 202, 203 and a hash value 201 of a previous block (parent block). A hash value of the block in question is calculated by performing a hash value calculation 301 for the block on a value based on the registration information 202, 203 and information 205 including the hash value 201 of the previous block. Note that a hash value of the block in question is not necessary to be calculated using all the registration information 202 and 203 and may be calculated using a value based on the registration information 202 and 203. A calculated hash value of the block in question is not registered in the block but is used as a hash value 211 of the previous block when generating the next block 210. In this way, by calculating a hash value of the block in question, for each block, using a hash value of the previous block, it is possible to make up a blockchain in which blocks that are difficult to be tampered with are concatenated. As a result, it is possible to register registration information that is difficult to be tampered with in each block.

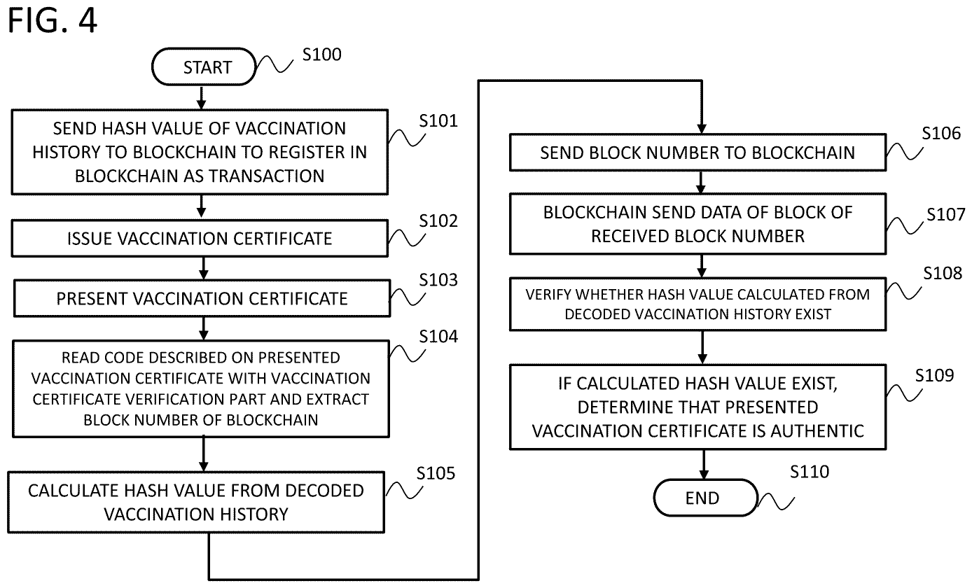

Next, an example of a schematic operation of the vaccination certificate verification system 100 according to the first example embodiment of the present invention will be described. FIG. 4 is a flowchart illustrating an example of a schematic operation of a vaccination certificate verification system 100 according to the first example embodiment of the present invention. The process starts at step S100.

In step S101, a doctor administers a prescribed vaccine to a person who wishes to be vaccinated. First, a doctor (medical institution) or an administrative agency or the like inputs a vaccination history 10 of a vaccinated person 120 into the vaccination history registration part 111. The vaccination history registration part 111, for example, sends a hash value of a following vaccination history 10 to a blockchain 150 to register in a blockchain 150 as a transaction. A person who registers a vaccination history in the vaccination history registration part 111 may be a doctor (medical institution) who administered a vaccine, or it may be an entity different from the doctor (medical institution), such as an administrative agency, that has received the vaccination history from a doctor (medical institution). In addition, the information registered as a block in a blockchain 150 includes not only a hash value of a vaccination history 10, but also a hash value for a transaction from any other third party and other block (parent block) that has been registered in a blockchain 150 immediately before the registration. Accordingly, it becomes possible to register a vaccination history 10 in a way which makes it difficult to be tampered with, and it is possible to keep a vaccination history 10 itself be confidential from a general viewer.

A vaccination history 10 may include, for example, the following information.
information on a vaccinated person (a person who was vaccinated) (vaccinated person ID),
information on a doctor (or a medical institute) who administered a vaccine (doctor ID)
a number of vaccinations,
a date and a time of vaccination,
a type of a vaccine being administered, and
a manufacturer of a vaccine, a manufacture date, and a lot number.

Next, in step S102, a vaccination history registration part 111 issues a vaccination certificate 121 including an above vaccination history 10 to a vaccinated person 120. Note, after a vaccination history has been registered to a blockchain 150, the vaccination history registration part 111 issues the vaccination certificate 121.

For example, a vaccination certificate 121 may include following information. Note, information, such as a vaccination history 10, described in a vaccination certificate 121 is assumed not to be hashed.
above vaccination history 10 (for conformation by a person who received a certificate)
a code (for example a QR (Quick Response) code (a QR code (Registered Trademark)) in which a block number of a block including
a hash value of the vaccination history 10 registered in a blockchain 150 is encoded as information regarding a vaccination history,
a person who received the vaccination certificate 121,
an issuance date of the vaccination certificate 121,
an issuer of the vaccination certificate 121, and
a signature of the issuer of the vaccination certificate 121.

Note, when a vaccinated person 120 has been vaccinated multiple times, a vaccination certificate 121 may include information of a last vaccination history 10, or, in addition, all of or some of information of each previous vaccination history 10 and so on. Furthermore, in a case where a different doctor (medical institute) administers a vaccine, the same vaccination history registration part 111 may register a hash value of each vaccination history 10 as a transaction (registration information) in the blockchain 150 and issue each vaccination certificate 121. In this case, even if a doctor (medical institute) who administered vaccine is different, all the vaccination histories 10 can be described in a vaccination certificate 121.

Next, in step S103, in a case where it is necessary to present a vaccination certificate 121 to a service provider 130 for receiving a service, a person who receives a service (a vaccinated person 120) presents a vaccination certificate 121 to the service provider 130 at the time of receiving a service from the service provider 130 (restaurant, movie theater, and so on). Note, if necessary, a service provider 130 can request to present an ID document at the same time. Note, the service provider 130 can initially check visually whether the ID document matches a person to whom the vaccination certificate 121 has been issued (a vaccinated person 120).

Next, in step S104, a service provider 130 reads a code (for example a QR code) described on a presented vaccination certificate 121 with a vaccination certificate verification part 131 including a reader and a vaccination certificate application and the vaccination certificate application decodes the QR code to extract a block number of a block which includes a vaccination history 10 and a hash value of the vaccination history 10 registered in a blockchain 150.

Note, a vaccination certificate verification part 131 may be configured by a smart phone, a mobile information terminal or the like which includes a camera part for reading a QR code, a QR code reading application, and a vaccination certificate application.

Next, in step S105, the vaccination certificate verification part (a vaccination certificate application) 131 calculates a hash value from a decoded vaccination history 10.

Next, in step S106, the vaccination certificate verification part (the vaccination certificate application) 131 connected to a network 140 sends the block number to a blockchain 150 through the network 140.

Next, in step S107, the blockchain 150 sends data of a block of the received block number to the vaccination certificate verification part (a vaccination certificate application) 131.

Next, in step S108, the vaccination certificate verification part (the vaccination certificate application) 131 verifies whether the hash value calculated from the decoded vaccination history 10 exists in the data of the received block from the blockchain 150.

Next, in step S109, if the calculated hash value exists, the vaccination certificate verification part (the vaccination certificate application) 131 determines that the presented vaccination certificate 121 is authentic.

Next, in step S110, the process ends at step S110.

As described above, according to the first example embodiment. it is possible to provide a vaccination certificate verification system, a vaccination certificate verification method, and a program that contribute to making it possible to verify the authenticity of the vaccination certificate.

### Second Example Embodiment

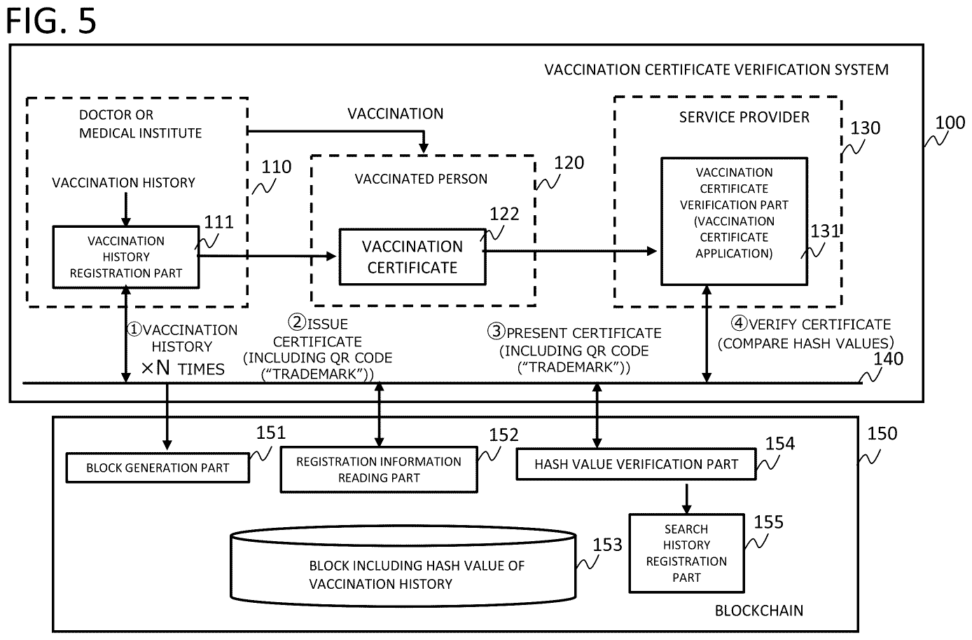

Next, a vaccination certificate verification system according to a second example embodiment of the present invention will be described with reference to drawings. FIG. 5 is a diagram illustrating an example of a schematic configuration of a vaccination certificate verification system according to the second example embodiment of the present invention. In FIG. 5, it is assumed that the components with the same reference signs as those in FIG. 2 are the same, and their description will be omitted.

The example of the schematic configuration of the vaccination certificate verification system according to the second example embodiment of the present invention as shown in FIG. 5 is such that a hash value verification part 154 and a search history registration part 155 are added to the schematic configuration of the vaccination certificate verification system according to the first example embodiment of the present invention as shown in FIG. 2.

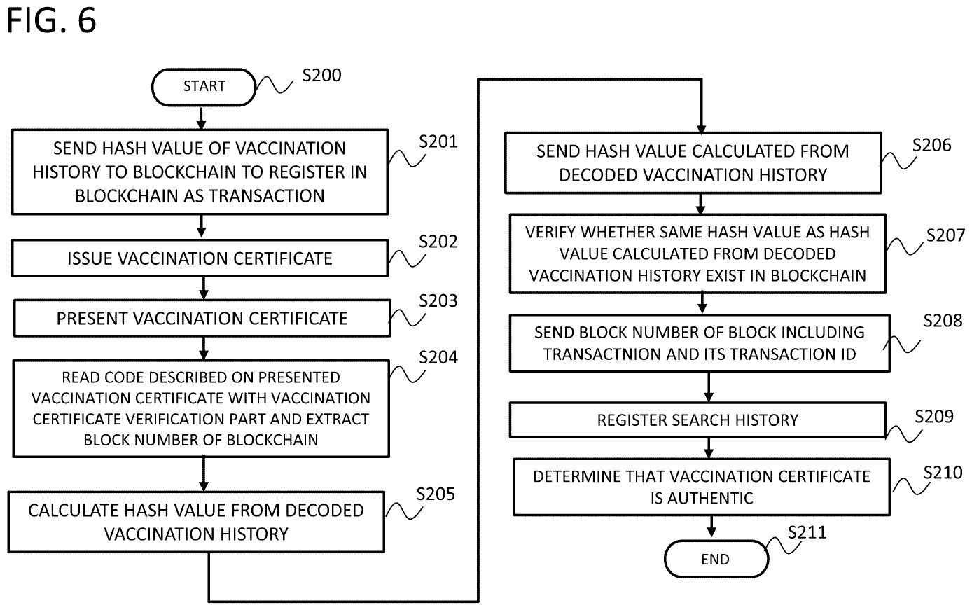

Next, an example of a schematic operation of the vaccination certificate verification system 100 according to the second example embodiment of the present invention will be described with reference to drawings. FIG. 6 is a flowchart illustrating an example of a schematic operation of a vaccination certificate verification system according to the second example embodiment of the present invention. The process starts at step S200.

Note, steps S200 to S205 of the schematic operation of the vaccination certificate verification system 100 according to the second example embodiment of the present invention are the same as the steps S100 to S105 according to the first example embodiment described with reference to FIG. 4, and their description will be omitted.

For steps S206 to S209, the vaccination certificate verification system 100 according to the second example embodiment operates as described below.

In step S206, the vaccination certificate verification part (a vaccination certificate application) 131 connected to a network 140 sends a hash value calculated from the decoded vaccination history to the blockchain 150 through the network 140. Note, the vaccination certificate verification part 131 may sends a block number of the blockchain 150 in which a vaccination history is recorded along with a hash value calculated from the decoded vaccination history.

Next, in step S207, a hash value verification part 154 in the blockchain 150 verifies whether the same hash value as the hash value calculated from the decoded vaccination history 10 exists in the blockchain 150.

Next, in step S208, the hash value verification part 154 in the blockchain 150 sends a block number of a block including a transaction in which the hash value exists and the transaction ID of the transaction to the vaccination certificate verification part (the vaccination certificate application) 131 if the same hash value as the hash value calculated from the decoded vaccination history 10 exists in the blockchain 150. Note, in a case where a block number of a blockchain 150 in which a vaccination history is recorded is also received along with a hash value, the blockchain 150 can verify whether the same hash value as the hash value calculated from the decoded vaccination history exists in the blockchain 150 in a higher speed.

Next, in step S209, furthermore, the blockchain 150 registers that the blockchain 150 has searched the hash value and has sent a transaction ID and a block number in which the hash value exists to the vaccination certificate verification part (the vaccination certificate application) 131, along with information identifying the vaccination certificate verification part (the vaccination certificate application) 131 in the blockchain 150 as a search history. It is possible to make tampering this search history difficult by recording it as one transaction in the blockchain 150. As a result, a vaccination certificate 121 can be presented to a service provider 130, it is registered that the service provider 130 acquired data of the vaccination certificate 121 whereby it is possible to suppress unauthorized use of the vaccination certificate 121 by the service provider 130.

Next, in step S210, when the vaccination certificate verification part (the vaccination certificate application) 131 receives a block number and a transaction ID, the vaccination certificate verification part (the vaccination certificate application) 131 determines that a hash values of the vaccination history in the vaccination certificate exists in the in the blockchain and determines that the presented vaccination certificate is authentic.

Next, the process ends at step S211.

As described above, according to the second example embodiment. it is possible to provide a vaccination certificate verification system, a vaccination certificate verification method, and a program that contribute to making it possible to verify the authenticity of the vaccination certificate.

Furthermore, according to the second example embodiment, it is possible to suppress unauthorized use of the vaccination certificate 121 by the service provider 130 by recording the search history in the blockchain 150.

### Third Example Embodiment

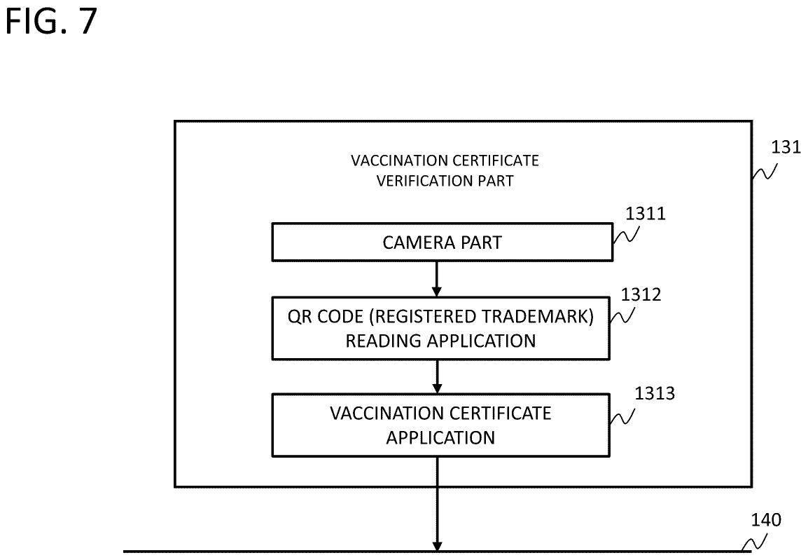

Next, a vaccination certificate verification system according to a third example embodiment of the present invention will be described with reference to drawings. FIG. 7 is a diagram illustrating an example of a configuration of a vaccination certificate verification part 131 according to the third example embodiment of the present invention. In FIG. 7, it is assumed that the components with the same reference signs as those in FIG. 2 are the same.

A vaccination certificate verification part 131 as shown in FIG. 7 includes a camera part 1311 for reading a QR code, a QR code reading application 1312, and a vaccination certificate application 1313.

A camera part 1311 and a QR code reading application 1312 make up a QR code reader. In step S104 of a flowchart illustrating an example of a schematic operation of a vaccination certificate verification system according to the first example embodiment of the present invention as shown in FIG. 4, a service provider 130 reads a code (for example a QR code) described on a presented vaccination certificate 121 with the camera part 1311 and the QR code reading application 1312, and the vaccination certificate application 1313 decodes the QR code to extract a vaccination history 10 and a block number of a block in which a hash value of the vaccination history 10 registered in a blockchain 150. Note, in a case where information other than a QR code is used as information regarding a vaccination history, a reading application according to information to be read can be used in place of the QR code reading application 1312.

According to the third example embodiment of the present invention, it is possible to make up a vaccination certificate verification part 131 of the first example embodiment.

Furthermore, a vaccination certificate verification part 131 according to the third example embodiment of the present invention may be configured to send a hash value calculated from a decoded vaccination history to a blockchain 150 through a network 140 in the same way as described in the second example embodiment.

The example embodiments of the present invention have been described as above, however, the present invention is not limited thereto. Further modifications, substitutions, or adjustments can be made without departing from the basic technical concept of the present invention. For example, the configurations of the network and the elements and the representation modes of the message or the like illustrated in the individual drawings are merely used as examples to facilitate the understanding of the present invention. Thus, the present invention is not limited to the configurations illustrated in the drawings. In addition, “A and/or B” signifies at least one of A or B.

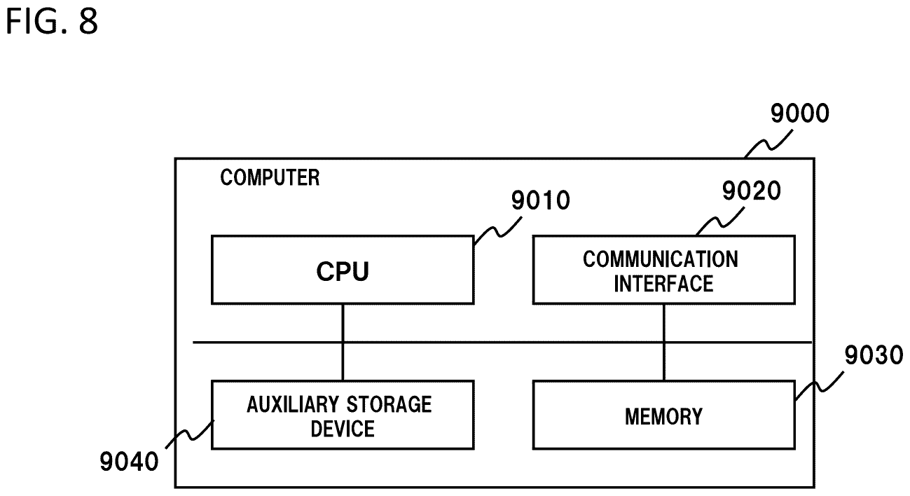

The procedures illustrated in the above first to the third example embodiment can each be realized by a program that causes a computer (9000 in FIG. 8), functioning as the corresponding vaccination certificate verification system or the vaccination certificate verification part to realize functions of a vaccination certificate verification system or a vaccination certificate verification part. The computer is, for example, configured to include a central processing unit (CPU) 9010, a communication interface 9020, a memory 9030, and an auxiliary storage device 9040 in FIG. 8. That is, the CPU 9010 in FIG. 8 executes a control program for a vaccination certificate verification system or a vaccination certificate verification part and executes a process for updating various calculation parameters stored in the auxiliary storage device 9040, etc.

The memory 9030 is a random-access memory (RAM), a read-only memory (ROM), or the like.

That is, the individual part (processing means or function) of the vaccination certificate verification system or the vaccination certificate verification part according to any one of the above first to the third example embodiments can be realized by a computer program that causes a processor of the above computer, using corresponding hardware, to execute a corresponding process described above.

Finally, suitable modes of the present invention will be summarized.

### Mode 1

(See the vaccination certificate verification system according to the above first aspect)

### Mode 2

The vaccination certificate verification system according to mode 1 is preferable that,
wherein a block number of a block of the blockchain in which the vaccination history is registered is encoded in the information regarding the history; and
wherein
the vaccination certificate verification part reads information regarding the history, decodes the vaccination history and the block number, calculates information identifying the decoded vaccination history and sends the decoded block number to the blockchain;
the vaccination certificate verification part receives data of a block of the block number from the blockchain; and
the vaccination certificate verification part determines that the vaccination certificate is authentic if the same information as the information identifying the decoded vaccination history exists in the block of the block number received from the blockchain.

### Mode 3

The vaccination certificate verification system according to mode 1 is preferable that,
wherein
the vaccination certificate verification part reads information regarding the history, decodes the vaccination history, calculates information identifying the decoded vaccination history and sends the information identifying the decoded vaccination history to the blockchain;
the vaccination certificate verification part receives a block number of a block including a transaction which includes the same information as the information identifying the decoded vaccination history and a transaction ID of the transaction from the blockchain if it has been verified that the same information as the information identifying the decoded vaccination history exists in the blockchain; and
the vaccination certificate verification part determines that the vaccination certificate is authentic when the vaccination certificate verification part receives the block number and the transaction ID.

### Mode 4

A blockchain connected to the vaccination certificate verification system according to mode 3, wherein the blockchain registers that the block number and the transaction ID has been sent to the vaccination certificate verification system.

### Mode 5

(See the vaccination certificate verification method according to the above second aspect)

### Mode 6

The vaccination certificate verification method according to mode 5, wherein a block number of a block of the blockchain in which the vaccination history is registered is encoded in the information regarding the history; and it is preferable to comprise:
a step of reading information regarding the history, decoding the vaccination history and the block number, calculating information identifying the decoded vaccination history and sending the decoded block number to the blockchain by the vaccination certificate verification part;
a step of receiving data of a block of the block number from the blockchain by the vaccination certificate verification part; and
a step of determining that the vaccination certificate is authentic if the same information as the information identifying the decoded vaccination history exists in the block of the block number received from the blockchain by the vaccination certificate verification part.

### Mode 7

The vaccination certificate verification method according to mode 5 is preferable to comprise:
a step of reading information regarding the history, decoding the vaccination history and the block number, calculating information identifying the decoded vaccination history and sending the information identifying the decoded vaccination history to the blockchain by the vaccination certificate verification part;
a step of receiving a block number of a block including a transaction which includes the same information as the information identifying the decoded vaccination history and a transaction ID of the transaction from the blockchain if it has been verified that the same information as the information identifying the decoded vaccination history exists in the blockchain by the vaccination certificate verification part; and
a step of determining that the vaccination certificate is authentic when the vaccination certificate verification part receives the block number and the transaction ID by the vaccination certificate verification part.

### Mode 8

(See the program according to the above third aspect)

### Mode 9

The program according to mode 8 is preferable that, wherein a block number of a block of the blockchain in which the vaccination history is registered is encoded in the information regarding the history; and causing the computer to execute processings of:
reading information regarding the history, decoding the vaccination history and the block number, calculating information identifying the decoded vaccination history and sending the decoded block number to the blockchain;
receiving data of a block of the block number from the blockchain; and
determining that the vaccination certificate is authentic if the same information as the information identifying the decoded vaccination history exists in the block of the block number received from the blockchain.

### Mode 10

The program according to mode 8 is preferable to cause the computer to execute processings of:
reading information regarding the history, decoding the vaccination history, calculating information identifying the decoded vaccination history and sending the information identifying the decoded vaccination history to the blockchain;
receiving a block number of a block including a transaction which includes the same information as the information identifying the decoded vaccination history and a transaction ID of the transaction from the blockchain if it has been verified that the same information as the information identifying the decoded vaccination history exists in the blockchain; and
determining that the vaccination certificate is authentic when the block number and the transaction ID are received.
Note, the above modes 5 and 8 can be expanded in the same way as mode 1 is expanded to mode 4.

The disclosure of each of the above PTLs and an NPL is incorporated herein by reference thereto. Modifications and adjustments of the example embodiments or examples are possible within the scope of the overall disclosure (including the claims) of the present invention and based on the basic technical concept of the present invention. Various combinations or selections of various disclosed elements (including the elements in each of the claims, example embodiments, examples, drawings, etc.) are possible within the scope of the disclosure of the present invention. That is, the present invention of course includes various variations and modifications that could be made by those skilled in the art according to the overall disclosure including the claims and the technical concept. The description discloses numerical value ranges. However, even if the description does not particularly disclose arbitrary numerical values or small ranges included in the ranges, these values and ranges should be deemed to have been specifically disclosed.

### REFERENCE SIGNS LIST

10 vaccination history
100 vaccination certificate verification system
110 doctor or medical institute
111 vaccination history registration part
120 vaccinated person
121 vaccination certificate
130 service provider
131 vaccination certificate verification part
140 network
150 blockchain
151 block generation part
152 registration information reading part
153 block including hash value of vaccination history
154 hash value verification part
155 search history registration part
200, 210 block
201, 211, 221 hash value of previous block
202, 203, 212, 213 registration information
205, 215 information including hash value 201 of previous block
301, 311 hash value calculation for block
1311 camera part
1312 QR code reading application
1313 vaccination certificate application
9000 computer
9010 CPU
9020 communication interface
9030 memory
9040 auxiliary storage device

What is claimed is:

1. A vaccination certificate verification system, comprising:
at least a processor; and
a memory in circuit communication with the processor,
wherein the processor is configured to execute program instructions stored in the memory to perform:
registering information identifying a vaccination history in a blockchain and issuing a vaccination certificate including information regarding the vaccination history after registration of the vaccination history; and
determining that the vaccination certificate is authentic in a case where information identifying the vaccination history included in information regarding the history in the vaccination certificate exists in the blockchain.

2. The vaccination certificate verification system according to claim 1,
wherein a block number of a block of the blockchain in which the vaccination history is registered is encoded in the information regarding the history; and
wherein the processor is configured to execute the program instructions to implement:
reading information regarding the history, decoding the vaccination history and the block number, calculating information identifying the decoded vaccination history and sending the decoded block number to the blockchain;
receiving data of a block of the block number from the blockchain; and
determining that the vaccination certificate is authentic if the same information as the information identifying the decoded vaccination history exists in the block of the block number received from the blockchain.

3. The vaccination certificate verification system according to claim 1,
wherein the processor is configured to execute the program instructions to implement:
reading information regarding the history, decoding the vaccination history, calculates information identifying the decoded vaccination history and sending the information identifying the decoded vaccination history to the blockchain;
receiving a block number of a block including a transaction which includes the same information as the information identifying the decoded vaccination history and a transaction ID of the transaction from the blockchain if it has been verified that the same information as the information identifying the decoded vaccination history exists in the blockchain; and
determining that the vaccination certificate is authentic when receiving the block number and the transaction ID.

4. (canceled)

5. A vaccination certificate verification method performed in by a vaccination certificate verification system-including a vaccination history comprising:
registering information identifying a vaccination history in a blockchain and issuing a vaccination certificate including information regarding the vaccination history after registration of the vaccination history; and
determining that the vaccination certificate is authentic in a case where information identifying the vaccination history included in information regarding the history in the vaccination certificate exists in the blockchain.

6. The vaccination certificate verification method according to claim 5,
wherein a block number of a block of the blockchain in which the vaccination history is registered is encoded in the information regarding the history; and comprising:
reading information regarding the history, decoding the vaccination history and the block number, calculating information identifying the decoded vaccination history and sending the decoded block number to the blockchain;
receiving data of a block of the block number from the blockchain; and
determining that the vaccination certificate is authentic if the same information as the information identifying the decoded vaccination history exists in the block of the block number received from the blockchain.

7. The vaccination certificate verification method according to claim 5, comprising:
reading information regarding the history, decoding the vaccination history and the block number, calculating information identifying the decoded vaccination history and sending the information identifying the decoded vaccination history to the blockchain;
receiving a block number of a block including a transaction which includes the same information as the information identifying the decoded vaccination history and a transaction ID of the transaction from the blockchain if it has been verified that the same information as the information identifying the decoded vaccination history exists in the blockchain; and
determining that the vaccination certificate is authentic when the vaccination certificate verification part receives the block number and the transaction ID.

8. A computer-readable non-transitory recording medium recording a program, the program, causing a computer to execute a processing of:
registering information identifying a vaccination history in a blockchain; and
determining that the vaccination certificate is authentic in a case where information identifying a vaccination history included in information regarding the history in a vaccination certificate including information regarding the vaccination history issued after registration of the vaccination history exists in the blockchain.

9. The medium program according to claim 8,
wherein a block number of a block of the blockchain in which the vaccination history is registered is encoded in the information regarding the history; and the program causing the computer to execute processings of:
reading information regarding the history, decoding the vaccination history and the block number, calculating information identifying the decoded vaccination history and sending the decoded block number to the blockchain;
receiving data of a block of the block number from the blockchain; and
determining that the vaccination certificate is authentic if the same information as the information identifying the decoded vaccination history exists in the block of the block number received from the blockchain.

10. The medium according to claim 8, the program causing the computer to execute processings of:
reading information regarding the history, decoding the vaccination history, calculating information identifying the decoded vaccination history and sending the information identifying the decoded vaccination history to the blockchain;
receiving a block number of a block including a transaction which includes the same information as the information identifying the decoded vaccination history and a transaction ID of the transaction from the blockchain if it has been verified that the same information as the information identifying the decoded vaccination history exists in the blockchain; and
determining that the vaccination certificate is authentic when the block number and the transaction ID are received.
